(19) United States
(12) Patent Application Publication
(10) Pub. No.: US 2007/0071781 A1
(43) Pub. Date: Mar. 29, 2007

(54) Compositions for rupturable capsules and methods of making the same

(75) Inventors: Richard C. Louys JR., Iron Mountain, MI (US); Brian LaVigne, Kingsford, MI (US); Bob Flugstad, Iron Mountain, MI (US); David Canfield, Seminole, FL (US)

Correspondence Address: MICHAEL BEST & FRIEDRICH, LLP, 100 E WISCONSIN AVENUE, MILWAUKEE, WI, 53202, US

(73) Assignee: Nelson Technologies, Inc., Kingsford, MI (US)

(21) Appl. No.: 11/521,085

(22) Filed: Sep. 14, 2006

Related U.S. Application Data
(60) Provisional application No. 60/718,424, filed on Sep. 19, 2005.
(60) Provisional application No. 60/719,994, filed on Sep. 23, 2005.

(51) Int. Cl. A61K 8/02; A01N 25/00

(52) U.S. Cl. 424/401; 424/405

(57) ABSTRACT

A rupturable capsule that includes a capsule containing a composition is disclosed. Also disclosed are compositions that include silica, oil, a bioorganic filler, colorant, fragrance, and a combination thereof. A method of imparting a colorant, a fragrance, and a combination thereof to an object, that includes rupturing a rupturable capsule on the object, is provided.

### CROSS-REFERENCE TO RELATED APPLICATIONS

This application claims priority to U.S. Provisional Applications Nos. 60/718,424 and 60/719,994, filed on Sep. 19, 2005 and Sep. 23, 2005, respectively, each of which is hereby fully incorporated by reference.

### BACKGROUND OF THE INVENTION

Rupturable capsules may be used for a broad array of applications. For instance, they may be used in everything from drug delivery systems to board games. They may also be used in paintball, BB, and marking pellet applications. In addition, they may be used to decorate clothing, or attract or deter animals.

A wide variety of compositions are used in rupturable capsules. More particularly, dipropylene glycol and different oils may be used in various applications of rupturable capsules. Over time, though, using diproplyene glycol and other oils can be cost prohibitive. Additionally, the viscosity of the composition may vary greatly according to the viscosity properties desired. New ways to improve the quality of rupturable capsules and the economics of production are sought.

### SUMMARY OF THE INVENTION

In one embodiment, the present invention provides a rupturable capsule that includes a capsule containing a composition comprising precipitated silica, an oil, and at least one of a colorant, a fragrance, and a combination thereof.

In another embodiment, the invention provides a method of imparting at least one of a colorant, fragrance, and a combination thereof to an object that includes rupturing a rupturable capsule on the object, the capsule comprising precipitated silica, an oil, and at least one of a color, fragrance, and a combination thereof.

The invention further provides a rupturable capsule that includes a capsule containing a composition comprising a bioorganic filler and silica.

In another embodiment, the invention provides a method of imparting at least one of a colorant, fragrance, and a combination thereof to an object that includes rupturing a rupturable capsule on the object, the capsule comprising a bioorganic filler, silica, an oil, and at least one of a color, fragrance, and a combination thereof.

Other aspects of the invention will become apparent by consideration of the detailed description. Before embodiments of the invention are explained in detail, it is to be understood that the invention is not limited in its application to the details of the compositions set forth in the following description. The invention is capable of other embodiments and of being practiced or being carried out in various ways. Also, it is understood that the phraseology and terminology used herein is for the purpose of description and should not be regarded as limiting.

### DETAILED DESCRIPTION OF THE PREFERRED EMBODIMENTS

This invention relates to a novel rupturable capsule that contains a composition that may comprise at least one of a colorant, oil, thickener or filler and emulsifier. It further relates to a method of imparting a color, a fragrance and a combination thereof to or near an object. The compositions of the present invention may be used in paintball, BB, and marking pellet applications and with other capsules that rupture or are specially designed not to rupture upon impact. The compositions may also be used to decorate clothing, or attract or deter animals.

The compositions may be used as fill materials encapsulated in a gelatin capsule, which capsule may be impact-rupturable. Gelatin capsules suitable for use with compositions of this invention may be formulated in accordance with conventional techniques for making filled, seamed or seamless, soft elastic gelatin capsules containing a fill material. See. e.g., Ebert, W. R., “Soft elastic gelatin capsules: a unique dosage form,” Pharmaceutical Tech., October 1977; Stanley, J. P., “Soft Gelatin Capsules,” The Theory and Practice of Industrial Pharmacy, 359-84 (Lea & Febiger ed. 1970). Optionally, the gelatin shell may contain preservatives such as mixed parabens, ordinarily methyl or propyl parabens, in about 4:1 ratio. The parabens may be incorporated in the shell formulation in minor proportions as compared to the total weight of the shell formulation. Conventional gelatin capsules utilize gelatin having a bloom value of about 140-200 although this amount may be varied. Using conventional techniques, the gelatin composition is mixed and melted under vacuum conditions. The capsules may be simultaneously formed and filled using a conventional method and apparatus such as those using rotary die process, as disclosed, for example, in U.S. Pat. Nos. 1,970,396; 2,288,327; and 2,318,718, which patents are herein fully incorporated by reference.

The term colorant, as used herein, is a compound that imparts color to surrounding materials. A variety of colors can be used as the colorant of the inventive composition. The liquid colorant composition and pigments can be colored red, magenta, pink, yellow, blue, orange, purple, violet, green, and the like. Typically, highly visible colors are used, including fluorescent versions of the colors. Colorants may be liquid, powders or solids.

Compositions of the invention include colorant formulations which are liquid and may be wiped from a surface on which they are dispersed. Liquid colorant compositions of the invention include solutions, suspensions or any combination thereof. The term “liquid” when used to describe the colorant compositions of the invention, is meant to include any non-solid physical state of the composition capable of dispersing or spreading onto a given surface, i.e., in both its highly flowable state as well as semi-solid viscous and “smearable” state.

Colorants suitable for use in the invention include pigments and dyes. A dye, as used herein, refers to a water-soluble compound that imparts color to the solution in which it is soluble. A pigment, as used herein, refers to a compound that imparts color to other materials and which is water-insoluble.

Aqueous or water-soluble dyes which can be used in the invention include, but are not limited to, FD&C Yellow # 5, FD&C Yellow #6, FD&C Red #40, FD&C Blue #1, (each available from Sensient Colors Inc., St. Louis, Mo.); FD&C Red #33, FD&C Red #28 (each available from Noveon Hilton Davis, Inc., Cincinnati, Ohio); FD&C Green #3, FD&C Blue #2, FD&C Red #3 (each available from Monarch Food Colors, High Ridge, Mo.), D56020 Chromatint Pyranine, Chromatint Black WSY Liquid 0073 (each available from Chromatech, Inc., Canton, Mich.), SYNOX HB-1094, (each available from Hoover Color Corp., Hiwassee, Va.).

The aqueous or water-soluble dye can be present in an amount sufficient to afford the composition with apparent coloration. The dye can be present in an amount ranging from at least about 0.01%, particularly at least about 0.04%, more particularly about 0.06%, and even more particularly at least about 0.08% by weight of the total composition. The dye may also be present in an amount that is less than about 10%, particularly less than about 5%, more particularly less than about 1%, and even more particularly less than about 0.2% by weight of the total composition.

Pigments suitable for use in compositions of the invention include titanium dioxide, such as Ti Pure CR800 (Kerr-McGee Chemical LLC, Oklahoma City, Okla.), Fluorescent Blue, Fluorescent Violet, Fluorescent Pink, Fluorescent Chartreuse, Fluorescent Orange, Fluorescent Green (each available from Dayglo, Cleveland, Ohio); Mearlin Card Silver BN001, Mearlin Card Gold BN002 (each available from Engelhard Corp., Peekskill, N.Y.); Iriodin 215 Rutile Red Pearl, Iriodin 153 Flash Pearl, Iriodin 123 Bright Lustre Sat in, Iriodin 225 Rutile Blue Pearl, Iriodin 502 Red Brown Pigment, Iriodin 119 Plar White (each available from EMD Chemicals Inc. Darmstadt, Germany), and Black Iron Oxide (available from Hoover Color Corp., Hiwassee, Va.).

The pigment can be present in an amount sufficient to afford the composition with apparent coloration. The pigment is suitably present up to 0.01%, 0.02%, 0.05%, 0.1%, 0.15%, 1.5%, 2%, 2.5%, 3%, 4%, or 5% by weight of the total composition. The pigment can be present in an amount ranging from at least about 0.01%, particularly at least about 0.05%, more particularly at least about 0.08%, and even more particularly at least about 0.1% by weight of the total composition. The pigment may also be present in an amount that is less than about 20%, particularly less than about 10%, more particularly less than about 7%, and even more particularly less than about 5% by weight of the total composition.

Compositions of the invention may include one or more oils. Suitable oils include, for example, ethers or esters liquid at room temperature (20° C.), such as vegetable or mineral oils. However, solid or relatively high melting paraffins, esters, waxes or fats may also be used.

Suitable oils are esters of trihydric and more than trihydric alcohols, more particularly vegetable oils or vegetable triglycerides, for example soybean oil, sunflower oil, corn oil, safflower oil, olive oil, almond oil, peanut oil, flaxseed (linseed) oil, cotton oil, pumpkin seed oil, coconut oil, canola oil, rice oil and the like. In one embodiment, soybean oil is used (Chicago-Industrial Oils, Chicago, Ill.). Esters of pentaerythritol, for example with pelargonic acid or oleic acid are also suitably used. Other suitable oils include fish oil and castor oil.

The oil is suitably present in an amount ranging from at least about 50%, particularly at least about 60%, more particularly at least about 75%, and even more particularly at least about 90% by weight of the total composition. The oil may also be present in an amount that is less than about 99%, particularly less than about 98%, more particularly less than about 97%, and even more particularly less than about 96% by weight of the total composition.

Compositions of the invention may include one or more thickeners or fillers. As used herein, a thickener or filler refers to a compound that has the capacity to modify the texture or viscosity of the composition and/or adds bulk to the composition. The terms thickener and filler are used interchangeably herein. At least one filler is preferred in the composition. Suitable fillers include, but are not limited to, wax, silica, organic thickeners such as flours and starches, calcium carbonate, borax (hydrated sodium borate), clays, bentonite clay-based greases such as Bentone, talc (magnesium silicate), diatomaceous earth such as Celite®, cellulose ethers such as Bermocoll, barium sulfates such as Sparwite Barytes W-5 HB (Mountain Minerals Co. Ltd., Lethbridge, Alberta), and hydrated aluminum magnesium silicates such as Attagel® 50.

Suitable thickeners include waxes and hydrogenated vegetable oils. Suitable wax thickeners include, but are not limited to, beeswax, and paraffin wax. Wax thickeners can be present in an amount up to 1%, 2%, 5%, 10%, 15%, 20%, 25% or 30% by weight of the total composition, or from about 1% to about 2%, 5%,10%, 15%, 20%, 25% or 30% by weight of the total composition.

Suitable silica thickeners include, but are not limited to, SiO₂ compositions. Suitably, modified hydrous silicates, hydrated alumina silicates, fumed or colloidal silica or organic derivatives of hydrous magnesium aluminum silicates may be used. Suitable SiO₂ compositions may include, for example, Cabosil™ and HDKN-20. A particularly suitable silica thickener is the precipitated silica thixotrope thickener, Hi-Sil T-700 (PPG Industries, Inc., Pittsburgh, Pa.). Silica thickeners is suitably present in an amount of up to about 1%, 2%, 3%, 5%, 10%, 15%, 20%, 25%, 30% or 50% by weight of the total composition. The silica thickener is suitably present in an amount ranging from at least about 0.5%, particularly at least about 1%, more particularly at least about 1.5%, and even more particularly at least about 2.5% by weight of the total composition. The silica thickener may also be present in an amount that is less than about 20%, particularly less than about 15%, more particularly less than about 10%, and even more particularly less than about 5% by weight of the total composition.

An organic filler, as used herein, refers to a filler comprising carbon. Organic fillers include carbon-based bioorganic fillers. A bioorganic filler, as used herein, refers to a filler comprising one or more carbon-based polymers that is obtainable from a biological organism. Suitable bioorganic fillers include those obtained from plants, such as flours and starches. A flour, as used herein, refers to a starch-containing ground meal obtainable from a plant.

Any of a plurality of different starches or flours can be employed as a filler. Suitably, starches and flours useful in the invention may be obtained from cereals such as rice, maize, wheat, barley, oats, spelt, or rye. Other starches and flours useful in the invention may be obtained from, for example, potato, peas, arrowroot, soy, taro, cattail, chickpeas, chestnuts or cassava (tapioca). Suitable organic thickeners may also include flours or starch in a purified or processed form. Suitable processed starches include, for example, Hylon V or Hylon VII.

Flours suitable for use in the invention may include, but are not limited to, cake flour, cookie/cracker flour such as graham flour, first clear flour, all purpose flour, high gluten flour, pastry flour, self-rising flour, second clear flour, stuff grades flour, spring patent flour spring/winter blend flour, strong spring patent flour, unbleached or bleached flour, winter standard flour, durum flour, durum granular flour, semolina flour, durum first clear flour, extra fancy durum flour, specialty flours such as color-sorted flour, cracked wheat flour, farina, heavy bran flour, light bran flour, malted barley flour, soft wheat flour, tang flour, whole wheat flour, such as stone ground whole wheat flour, coarse or finely ground flour and vital wheat flour. In one embodiment, the flour may comprise bleached wheat flour (Conagra Inc., Omaha, Nebr.). In another embodiment, the flour may comprise corn starch.

Flour thickeners are suitably present in an amount up to about 15%, 20%, 25%, 30%, 40%, 50% or 60% by weight of the total composition. The flour is suitably present in an amount ranging from at least about 2%, particularly at least about 5%, more particularly at least about 10%, and even more particularly at least about 12% by weight of the total composition. The flour may also be present in an amount that is less than about 50%, particularly less than about 30%, more particularly less than about 25%, and even more particularly less than about 22% by weight of the total composition.

Generally, the relative amounts and proportions of the thickeners in the composition will vary according to the viscosity properties desired. A higher viscosity product is generally preferred by the consumer. The viscosity range for compositions containing dye may be from about 1,000 centipoises (cps) to about 10,000 cps. More particularly, the viscosity range for compositions containing dye may be from about 2,000 cps to about 5,000 cps. More particularly, the viscosity range for compositions containing dye may be from about 2,000 cps to about 3,000 cps, and even more particularly, right at about 2,500 cps. In another embodiment, the viscosity range for compositions containing pigment may be from about 1,000 cps to about 10,000 cps. More particularly, the viscosity range for compositions containing pigment may be from about 2,000 cps to about 5,000 cps. More particularly, the viscosity range for compositions containing pigment may be from about 2,000 cps to about 3,000 cps, and even more particularly, right at about 2,500 cps. In another embodiment, the viscosity range for compositions containing pigment may be from about 5,000 cps to about 15,000 cps. More particularly, the viscosity range for compositions containing pigment may be from about 7,500 cps to about 12,500 cps. More particularly, the viscosity range for compositions containing pigment may be from about 9,000 cps to about 11,000 cps, and even more particularly, right at about 10,000 cps. In yet another embodiment, the viscosity range for compositions containing pigment may be from about 10,000 cps to about 40,000 cps. More particularly, the viscosity range for compositions containing pigment may be from about 20,000 cps to about 30,000 cps. More particularly, the viscosity range for compositions containing pigment may be from about 22,500 cps to about 27,500 cps, and even more particularly, right at about 25,000 cps.

Additional ingredients can be used in accordance with the invention, provided the inventive attributes of the composition are maintained. Additional ingredients, such as opacifiers, emulsifiers, binding agents, fragrances, and preservatives may be added to the composition.

Emulsifiers suitable for use in the invention include, but are not limited to, S Maz™-80, S Maz™-60, Pluronic™ L31, Pluronic™ L92, tannins, lecithins, metatartric acid, calcium tartrate, alginic acid sodium, potassium, ammonium and calcium alginate, propylene glycol alginate, carrageenan, processes eucheuma seaweed, locust bean gum, gum acacia, gum Arabic, karaya gum, tara gum, gellan gum, sorbitol, polyethyoxylene stearate, polysorbates, pectin, gelatine, ammonium phosphatides, brominated vegetable oil, sucrose acetate isobutrate, glycerol esters of wood rosin, diphosphates, triphosphates, polyphosphates, beta-cyclodextrine, methylcellulose, hydroxypropyl celluloses, methyl ethyl cellulose, ethyl methyl cellulose, magnesium stearate, fatty acid salts (sodium, potassium, calcium or magnesium), mono and diglycerides of fatty acids, ethoxylated mono and diglycerides of fatty acids, esters of mono and diglycerides of fatty acids (including acetic acid, lactic acid, citric acid, tartaric acid, and diacetyltartaric acid esters of mono and diglycerides of fatty acids), sucroglycerides, sucrose, polypropylene glycol and polyglycerol esters of fatty acids, polyglycerol polyricinoleate, lactylated fatty acid esters of glycerol and polypropylene glycol, oxidized soya bean oil, diacotyl sodium sulfosuccinate, sodium and calcium stearoyl lactylate, stearyl tartrate, sorbitan monostearate, sorbitan tristearate, sorbitan monolaurate, sorbitan monolaurate, sorbitan monooleate, sorbitan monopalmitate, sodium aluminum phosphate, dimethylpolysiloxane and choline salts and esters.

The emulsifier can be present in an amount sufficient to provide a stable emulsion in accordance with expected storage and usage temperatures and conditions. The emulsifier is suitably present in an amount ranging from at least about 0.5%, particularly at least about 1%, more particularly at least about 2%, and even more particularly at least about 3% by weight of the total composition. The emulsifier may also be present in an amount that is less than about 20%, particularly less than about 15%, more particularly less than about 12%, and even more particularly less than about 7% by weight of the total composition.

Binding agents may be included in compositions of the invention to assist in maintaining a homogeneous suspension or solution. Suitable binding agents include, but are not limited to, silicas such as Hi-Sil™ T-700, Aerosil™ 200 and Cabosil™, gums and borax. Binding agents may be used in amounts effective to facilitate homogeneity of the solution or suspension under desired conditions.

Fragrances suitable for use in compositions of the invention include, but are not limited to, acacia, apple blossom, azalea, bluebell, carnation, clover, cornflower, frangipani, freesia, gardenia, geranium, honeysuckle, herbal flowers, hyacinth, iris, jasmine, jonquil, lavender, lilac, lily of the valley, lotus blossom, magnolia, mimosa, muguet, narcissus, orange blossom, powdery musk, poppy, rose, spring bouquet, sweet pea, verbena, violet, wild flower, wisteria, ylang, birch, bluegrass, blue spruce, Canadian spruce, cedarwood, evergreen, fir balsam, forest, fresh air, fresh meadows, juniper, mountain air, sea breeze, Siberian pine, blue spruce, ambrosia, green apple, red apple, sour apple, apricot, banana, berry, blueberry, boysenberry, black cherry, wild cherry, coconut, cranberry, grape, grapefruit, guava, honeydew, lemon, lime, orange, papaya, passion fruit, peach, peaches ‘n’ cream, pear, pineapple, plum, raspberry, fresh strawberry, strawberry jam, wild strawberry, tangerine, tropical fruit, watermelon, amber, cashmere, frankincense, musk, myrrh, oriental, patchouli, sandalwood, vetiver, amaretto, anisette, beer, burgundy, champagne, cream sherry, creme de cocoa, creme de menthe, Grand Marnier type, Hawaiian punch type, pina coloda, rum, scotch, almond, anise, apple cider, apple pie, baby powder, baked bread, brown sugar, butter, butter scotch, caramel, carrot, cheese, chili aroma, dark chocolate, milk chocolate, citronella, clover, coffee, cola, cookie, cucumber, egg nog, french vanilla, hazelnut, herbal (hemp), hickory smoke, honey, hot cocoa, ice cream, incense, leather, licorice, maple syrup, medicinal, menthol, mint, peanut butter, pecan, butter pecan, peppermint, pickles, pistachio, pizza, popcorn, root beer, sassafras, smoke deodorant, spearmint, spice mix, vanilla, walnut, wintergreen, bayberry, candy cane, Christmas tree, cranberry, gingerbread, hollyberry, mistletoe, pomander, pumpkin pie, allspice, bay, black pepper, caraway, cardamom, cassia, cinnamon, clove, coriander, curry, eucalyptus, fennel, ginger, kitchen spice, nutmeg, old spice type, oregano, pennyroyal, rosemary, sage, thyme, and perfumes (each available from Andrea Aromatics, Princeton, N.J.). Particularly suitable scents include corn, BuckLure, green apple, red apple, strawberry, orange, acorn and cedar scents (each available from Andrea Aromatics, Princeton, N.J.).

Suitably, fragrances may be used in amounts effective to provide sufficient scent to attract or deter an animal exposed to the composition comprising the scent. Fragrances may be present in an amount ranging from about 1% to about 50% by weight of the total composition. More particularly, fragrances may also be present in an amount ranging from about 1% to about 10% by weight of the total composition.

Preservatives suitable for use in compositions of the invention include, but are not limited to, methyl/propyl paraben, borax, citric acid and sodium benzoate. Preservatives may be used in amounts effective to preserve the composition over desired periods of time. More particularly, citric acid may be present in amounts less than about 1% by weight of the total composition.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. In particular, one of skill in the art will appreciate that the time for mixing is reliant on the size and thickness of each batch and will vary.

All patents and publications listed or described herein are incorporated in their entirety by reference.

### EXAMPLES

### Example 1

### Dye Formula

TABLE 1

| | lbs. | weight % |
|---|---|---|
| Vegetable Oil (Soybean) | 1618.2 | 79.93% |
| Flour | 304 | 15.02% |
| Silica (Hi-Sil ™ T-700) | 61 | 3.01% |
| Dye | 2.5 | 0.12% |
| Titanium dioxide (Ti Pure | 8.3 | 0.41% |
| CR800) | | |
| Water | 30.4 | 1.50% |
| | 2024.4 | 100.00% |

A dye formula composition was prepared in a 250 gallon batch using components described in Table 1. 1079.1 lbs of soybean oil (available from Chicago-Industrial Oils, Chicago, Ill.) were weighed off into a mixing vessel at room temperature. Mixing was started and 304 lbs of bleached white sifted wheat flour were slowly added over a 15 minute time period. The batch was allowed to mix for additional 10 minutes. While mixing, 61 lbs of precipitated silica thixotrope thickener (Hi-Sil™ T-700, available from PPG Industries, Inc., Pittsburgh, Pa.) were slowly added over a 15 minute period. 2.5 lbs of a yellow dye (available from Chromatech, Inc., Canton, Mich.), which had been pre-mixed with 30.4 lbs of water and slurried with 8.3 lbs of titanium dioxide (Ti Pure CR800, available from Kerr-McGee Chemical LLC, Oklahoma City, Okla.), was then added and the batch was mixed until blended to a uniform color. Mixing was stopped for approximately 10 minutes, before an additional 539 lbs of soybean oil were added to the batch. The batch was mixed for an additional 15 minutes until it was blended.

### Example 2

### Pigment Formula

TABLE 2

| | lbs. | weight % |
|---|---|---|
| Vegetable Oil (Soybean) | 1607.7 | 79.28% |
| Flour | 302 | 14.89% |
| Silica (Hi-Sil ™ T-700) | 60.6 | 2.99% |
| Fluorescent Green Pigment | 22.36 | 1.1% |
| Titanium dioxide (Ti Pure | 4.97 | 0.24% |
| CR800) | | |
| Water | 30.2 | 1.49% |
| | 2027.93 | 100.00% |

A pigment formula composition was prepared in a 250 gallon batch using components described in Table 2. 1072.1 lbs of soybean oil (available from Chicago-Industrial Oils, Chicago, Ill.) were weighed off into a mixing vessel at room temperature. Mixing was started and 302 lbs of bleached white sifted wheat flour were slowly added over a 15 minute time period. The batch was allowed to mix for additional 10 minutes. While mixing, 60.6 lbs of precipitated silica thixotrope thickener (Hi-Sil™ T-700, available from PPG Industries, Inc., Pittsburgh, Pa.) were slowly added over a 15 minute period. Pigments (4.96 lbs of Ti Pure CR800, available from Kerr-McGee Chemical LLC, Oklahoma City, Okla., and 22.357 lbs of fluorescent green, available from Dayglo, Cleveland, Ohio) were then added and the batch was mixed until it was blended to a uniform color. Mixing was stopped for approximately 10 minutes, before an additional 535.6 lbs of soybean oil were added to the batch. 30.2 lbs of water were added and the batch was mixed for an additional 15 minutes until it was blended.

### Example 3

### Thicker-Fill Dye Formula

TABLE 3

| | lbs. | weight % |
|---|---|---|
| Soybean Oil | 993.51 | 48.32% |
| Flour | 390.25 | 18.98% |
| Silica (Hi-Sil ™ T-700) | 82.73 | 4.02% |
| Soybean Oil | 548.63 | 26.68% |
| No. 8006 & 07797 Yellow Dye | 2.08 | 0.10% |
| Titanium dioxide (Ti Pure | 8.28 | 0.40% |
| CR800) | | |
| Water | 30.82 | 1.50% |
| | 2056.30 | 100.00% |

A thicker-fill dye formula composition is prepared in a 250 gallon batch using components described in Table 3. 993.51 lbs of soybean oil (available from Chicago-Industrial Oils, Chicago, Ill.) are weighed off into a mixing vessel at room temperature. Mixing is started and 390.25 lbs of bleached white sifted wheat flour are slowly added over a 15 minute time period. The batch is allowed to mix for additional 10 minutes. While mixing, 82.73 lbs of precipitated silica thixotrope thickener (Hi-Sil™ T-700, available from PPG Industries, Inc., Pittsburgh, Pa.) are slowly added over a 15 minute period. 2.08 lbs of a yellow dye (available from Chromatech, Inc., Canton, Mich.), which is pre-mixed with 30.4 lbs of water and slurried with 8.3 lbs of titanium dioxide (Ti Pure CR800, available from Kerr-McGee Chemical LLC, Oklahoma City, Okla.), is then added and the batch is mixed until blended to a uniform color. Mixing is stopped for approximately 10 minutes, before an additional 548.63 lbs of soybean oil are added to the batch. The batch is mixed for an additional 15 minutes until it was blended.

### Example 4

### Thicker-Fill Pigment Formula

TABLE 4

| | lbs. | weight % |
|---|---|---|
| Soybean Oil | 984.87 | 47.81% |
| Sifted Bleached Flour | 390.54 | 18.96% |
| Hi-Sil ™ T-700 | 82.90 | 4.02% |
| Titanium dioxide (Ti Pure CR800) | 3.97 | 0.19% |
| R6 GR9011 Fluorescent Green | 22.60 | 1.10% |
| Soybean Oil | 544.35 | 26.42% |
| Water | 30.90 | 1.50% |
| | 2060.13 | 100.00% |

A thicker-fill pigment formula composition is prepared in a 250 gallon batch using components described in Table 4. 984.87 lbs of soybean oil (available from Chicago-Industrial Oils, Chicago, Ill.) are weighed off into a mixing vessel at room temperature. Mixing is started and 390.54 lbs of bleached white sifted wheat flour are slowly added over a 15 minute time period. The batch is allowed to mix for additional 10 minutes. While mixing, 82.90 lbs of Precipitated Silica Thixotrope thickener (Hi-Sil™ T-700, available from PPG Industries, Inc., Pittsburgh, Pa.) are slowly added over a 15 minute period. Pigments (3.97 lbs of Ti Pure CR800, available from Kerr-McGee Chemical LLC, Oklahoma City, Okla., and 22.6 lbs of fluorescent green, available from Dayglo, Cleveland, Ohio) are then added and the batch is mixed until it is blended to a uniform color. Mixing is stopped for approximately 10 minutes, before an additional 544.35 lbs of soybean oil are added to the batch. 30.9 lbs of water is added and the batch was mixed for an additional 15 minutes until it is blended.

### Example 5

### Dye Formula

TABLE 5

| | lbs. | weight % |
|---|---|---|
| Vegetable Oil (Soybean) | 1865 | 95.54% |
| Silica (Hi-Sil ™ T-700) | 60 | 3.07% |
| Dye | 2.25 | 0.12% |
| Titanium dioxide (Ti Pure | 4.25 | 0.22% |
| CR800) | | |
| Water | 20.5 | 1.05% |
| | 1952 | 100.00% |

A dye formula composition was prepared in a 250 gallon batch using components described in Table 5. 1255 lbs of soybean oil (available from Chicago-industrial Oils, Chicago, Ill.) were weighed off into a mixing vessel at room temperature. While mixing, 60 lbs of precipitated silica thixotrope thickener (Hi-Sil™ T-700, available from PPG Industries, Inc., Pittsburgh, Pa.) were slowly added over a 15 minute period. 4.25 lbs of titanium dioxide (Ti Pure CR800, available from Kerr-McGee Chemical LLC, Oklahoma City, Okla.), were then added and the batch was mixed until blended to a uniform color. Mixing was stopped for approximately 10 minutes, before an additional 610 lbs of soybean oil were added to the batch. The batch was mixed until blended. 2.25 lbs of a yellow dye (available from Chromatech, Inc., Canton, Mich.), which had been pre-mixed with 20.5 lbs of water was added. The batch was mixed for an additional 15 minutes until it was blended.

### Example 6

### Pigment Formula

TABLE 6

| | lbs. | weight % |
|---|---|---|
| Vegetable Oil (Soybean) | 1864 | 95.28% |
| Silica (Hi-Sil ™ T-700) | 60 | 3.07% |
| Pigment - Fluorescent Chartreuse | 30 | 1.53% |
| Titanium dioxide (Ti Pure CR800) | 2.5 | 0.13% |
| | 1956.5 | 100.00% |

A pigment formula composition was prepared in a 250 gallon batch using components described in Table 6. 1255 lbs of soybean oil (available from Chicago-Industrial Oils, Chicago, Ill.) were weighed off into a mixing vessel at room temperature. Mixing was started and 60 lbs of precipitated silica thixotrope thickener (Hi-Sil™ T-700, available from PPG Industries, Inc., Pittsburgh, Pa.) were slowly added over a 15 minute period. Pigments (30 lbs of fluorescent chartreuse, available from Dayglo, Cleveland, Ohio and 2.5 lbs of Ti Pure CR800, available from Kerr-McGee Chemical LLC, Oklahoma City, Okla.) were then added and the batch was mixed until it was blended to a uniform color. Mixing was stopped for approximately 10 minutes, before an additional 610 lbs of soybean oil were added to the batch. The batch was mixed for an additional 15 minutes until it was blended.

### Example 7

### Thicker-Fill Dye Formula

TABLE 7

| | lbs. | weight % |
|---|---|---|
| Vegetable Oil (Soybean) | 1855 | 94.06% |
| Silica (Hi-Sil ™ T-700) | 90 | 3.01% |
| Dye | 2.25 | 0.11% |
| Titanium dioxide (Ti Pure CR800) | 4.25 | 0.22% |
| Water | 20.5 | 1.04% |
| | 1972 | 100.00% |

A dye formula composition is prepared in a 250 gallon batch using components described in Table 7. 1245 lbs of soybean oil (available from Chicago-Industrial Oils, Chicago, Ill.) are weighed off into a mixing vessel at room temperature. While mixing, 90 lbs of precipitated silica thixotrope thickener (Hi-Sil™ T-700, available from PPG Industries, Inc., Pittsburgh, Pa.) are slowly added over a 15 minute period. 4.25 lbs of titanium dioxide (Ti Pure CR800, available from Kerr-McGee Chemical LLC, Oklahoma City, Okla.), are then added and the batch is mixed until blended to a uniform color. Mixing is stopped for approximately 10 minutes, before an additional 610 lbs of soybean oil is added to the batch. The batch is mixed. 2.25 lbs of a yellow dye (available from Chromatech, Inc., Canton, Mich.), which is pre-mixed with 20.5 lbs of water is added. The batch is mixed for an additional 15 minutes until it is blended.

### Example 8

### Thicker-Fill Pigment Formula

TABLE 8

| | lbs. | weight % |
|---|---|---|
| Soybean Oil | 1854 | 92.4% |
| Hi-Sil ™ T-700 | 90 | 4.49% |
| Titanium dioxide (Ti Pure CR800) | 2.5 | 0.12% |
| Pigment - Fluorescent Chartreuse | 60 | 2.99% |
| | 2006.5 | 100.00% |

A thicker-fill pigment formula composition was prepared in a 250 gallon batch using components described in Table 8. 1245 lbs of soybean oil (available from Chicago-Industrial Oils, Chicago, Ill.) were weighed off into a mixing vessel at room temperature. Mixing was started and 90 lbs of precipitated silica thixotrope thickener (Hi-Sil™ T-700, available from PPG Industries, Inc., Pittsburgh, Pa.) were slowly added over a 15 minute period. Pigments (60 lbs of fluorescent chartreuse, available from Dayglo, Cleveland, Ohio and 2.5 lbs of Ti Pure CR800, available from Kerr-McGee Chemical LLC, Oklahoma City, Okla.) were then added and the batch was mixed until it was blended to a uniform color. Mixing was stopped for approximately 10 minutes, before an additional 610 lbs of soybean oil were added to the batch. The batch was mixed for an additional 15 minutes until it was blended.

### Example 9

### Dye Formula

TABLE 9

| | lbs. | weight % |
|---|---|---|
| Vegetable Oil (Soybean) | 1862 | 95.53617% |
| Silica (Hi-Sil ™ T-700) | 60 | 3.0785% |
| No. 8006 & 07797 Yellow Dye | 2.25 | 0.11544% |
| Titanium dioxide (Ti Pure CR800) | 4.25 | 0.21806% |
| Water | 10 | 1.05182% |
| | 1949 | 99.9999% |

A dye formula composition was prepared in a 250 gallon batch using components described in Table 9. 1862 lbs of soybean oil (available from Chicago-Industrial Oils, Chicago, Ill.) were weighed off into a mixing vessel at room temperature. While mixing, 60 lbs of precipitated silica thixotrope thickener (Hi-Sil™ T-700, available from PPG Industries, Inc., Pittsburgh, Pa.) were slowly added over a 15 minute period. 2.25 lbs of a yellow dye (available from Chromatech, Inc., Canton, Mich.), which had been pre-mixed with 10 lbs of water and slurried with 4.25 lbs of titanium dioxide (Ti Pure CR800, available from Kerr-McGee Chemical LLC, Oklahoma City, Okla.), was then added and the batch was mixed an additional 15 minutes until blended to a uniform color. The formulation was measured to have a viscosity of about 2500 cps.

### Example 10

### Pigment Formula

TABLE 10

| | lbs. | weight % |
|---|---|---|
| Vegetable Oil (Soybean) | 1853.5 | 95.61516% |
| Silica (Hi-Sil ™ T-700) | 45.0 | 2.32138% |
| R6 GR9011 Fluorescent Green | 27.5 | 1.41862% |
| Titanium dioxide (Ti Pure | 2.50 | 0.12896% |
| CR800) | | |
| Water | 10.0 | 0.51586% |
| | 1938.5 | 99.9998% |

A pigment formula composition was prepared in a 250 gallon batch using components described in Table 10. 1853.5 lbs of soybean oil (available from Chicago-Industrial Oils, Chicago, Ill.) were weighed off into a mixing vessel at room temperature. While mixing, 45 lbs of precipitated silica thixotrope thickener (Hi-Sil™ T-700, available from PPG Industries, Inc., Pittsburgh, Pa.) were slowly added over a 15 minute period. Pigments (2.50 lbs of Ti Pure CR800, available from Kerr-McGee Chemical LLC, Oklahoma City, Okla., and 27.5 lbs of fluorescent green, available from Dayglo, Cleveland, Ohio) were then added and the batch was mixed until it was blended to a uniform color. 10 lbs of water was added and the batch was mixed for an additional 15 minutes until it is blended. The formulation was measured to have a viscosity of about 2500 cps.

### Example 11

### Thicker-Fill Pigment Formula

TABLE 11

| | lbs. | weight % |
|---|---|---|
| Vegetable Oil (Soybean) | 1857 | 94.89013% |
| Silica (Hi-Sil ™ T-700) | 60 | 3.06591% |
| Pigment - Fluorescent | 27.5 | 1.40521% |
| Chartreuse | | |
| Titanium dioxide (Ti Pure | 2.5 | 0.12774% |
| CR800) | | |
| Water | 10.0 | 0.51098% |
| | 1957.0 | 99.99997% |

A thicker-fill pigment formula composition was prepared in a 250 gallon batch using components described in Table 11. 1857 lbs of soybean oil (available from Chicago-Industrial Oils, Chicago, Ill.) were weighed off into a mixing vessel at room temperature. While mixing, 60 lbs of Precipitated Silica Thixotrope thickener (Hi-Sil™ T-700, available from PPG Industries, Inc., Pittsburgh, Pa.) were slowly added over a 15 minute period. Pigments (2.50 lbs of Ti Pure CR800, available from Kerr-McGee Chemical LLC, Oklahoma City, Okla., and 27.5 lbs of fluorescent chartreuse, available from Dayglo, Cleveland, Ohio) were then added and the batch was mixed until it was blended to a uniform color. 10 lbs of water was added and the batch was mixed for an additional 15 minutes until it was blended. The formulation was measured to have a viscosity of about 10000 cps.

### Example 12

### Thicker-Fill Pigment Formula

TABLE 12

| | lbs. | weight % |
|---|---|---|
| Vegetable Oil (Soybean) | 1815 | 92.24881% |
| Silica (Hi-Sil ™ T-700) | 75 | 3.81193% |
| Pigment - Fluorescent | 55 | 0.12731% |
| Chartreuse | | |
| Titanium dioxide (Ti Pure | 2.5 | 2.179541% |
| CR800) | | |
| Water | 20.0 | 1.01651% |
| | 1967.505 | 99.99997% |

A thicker-fill pigment formula composition was prepared in a 250 gallon batch using components described in Table 12. 1815 lbs of soybean oil (available from Chicago-Industrial Oils, Chicago, Ill.) were weighed off into a mixing vessel at room temperature. Mixing was started and 75 lbs of precipitated silica thixotrope thickener (Hi-Sil™ T-700, available from PPG Industries, Inc., Pittsburgh, Pa.) were slowly added over a 15 minute period. Pigments (2.50 lbs of Ti Pure CR800, available from Kerr-McGee Chemical LLC, Oklahoma City, Okla., and 55 lbs of fluorescent chartreuse, available from Dayglo, Cleveland, Ohio) were then added and the batch was mixed until it was blended to a uniform color. 10 lbs of water was added and the batch was mixed for an additional 15 minutes until it was blended. The formulation was measured to have a viscosity of about 25000 cps.

### Example 13

### Fragrance Formula

TABLE 13

| | lbs. | weight % |
|---|---|---|
| Vegetable Oil (Soybean) | 1795.5 | 91.93548% |
| Silica (Hi-Sil ™ T-700) | 60.0 | 3.07219% |
| Pigment - Fluorescent | 55 | 2.81618% |
| Chartreuse | | |
| Titanium dioxide (Ti Pure | 2.505 | 0.12826% |
| CR800) | | |
| Water | 20.0 | 1.02406% |
| Green Apple fragrance | 20.0 | 1.02406% |
| | 1953 | 100.00% |

A fragrance formula composition was prepared in a 250 gallon batch using components described in Table 13. 1795.5 lbs of soybean oil (available from Chicago-Industrial Oils, Chicago, Ill.) were weighed off into a mixing vessel at room temperature. Mixing was started and 60.0 lbs of precipitated silica thixotrope thickener (Hi-Sil™ T-700, available from PPG Industries, Inc., Pittsburgh, Pa.) were slowly added over a 15 minute period. Pigments (2.505 lbs of Ti Pure CR800, available from Kerr-McGee Chemical LLC, Oklahoma City, Okla., and 55 lbs of fluorescent chartreuse, available from Dayglo, Cleveland, Ohio) were then added and the batch was mixed until it was blended to a uniform color. 20 lbs of water was added and the batch was mixed for an additional 15 minutes until it was blended. 20 lbs of green apple fragrance (available from Andrea Aromatics, Princeton, N.J.) was added and the batch was mixed for an additional 15 minutes until it was blended.

All of the compositions and methods disclosed herein can be made or executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are chemically related may be substituted for the agents described herein while the same or similar results would be achieved. All such similar substitutes and modifications apparent to those skilled in the art are deemed to be within the spirit, scope and concept of the invention.

We claim:

1. A rupturable capsule comprising:
a capsule containing a composition comprising:
precipitated silica;
an oil; and
at least one of a colorant, a fragrance, and a combination thereof.

2. The capsule of claim 1, wherein the oil includes at least one of the following: ethers, esters, paraffins, waxes, fats, and a combination thereof.

3. The capsule of claim 1, wherein the composition comprises a colorant and the colorant includes at least one of the following: pigments and dyes.

4. The capsule of claim 1, wherein the composition comprises a colorant.

5. The capsule of claim 1, wherein the composition comprises a fragrance.

6. The capsule of claim 5, wherein the fragrance includes at least one of the following: corn scent, apple scent, strawberry scent, orange scent, acorn scent, and cedar scent.

7. The capsule of claim 1, wherein the composition comprises a combination of a colorant and a fragrance.

8. The capsule of claim 1, wherein the composition further comprises at least one of the following: opacifiers, emulsifiers, binding agents, preservatives, and any combination thereof.

9. The capsule of claim 1, wherein the capsule is a gelatin capsule.

10. The capsule of claim 1, wherein the silica is present in an amount of about 1% to about 50% by weight of the total composition.

11. The capsule of claim 1, wherein the oil is present in an amount of about 50% to about 99% by weight of the total composition.

12. The capsule of claim 1, wherein the colorant is present in an amount of about 0.01% to about 20% by weight of the total composition.

13. The capsule of claim 1, wherein the fragrance is present in an amount of about 1% to about 10% by weight of the total composition.

14. A method of imparting at least one of a color, a fragrance, and a combination thereof to an object comprising:
rupturing a rupturable capsule on the object, the capsule comprising:
precipitated silica;
an oil; and
at least one of a color, a fragrance, and a combination thereof.

15. The method of claim 14, wherein the method decorates clothing.

16. The method of claim 14, wherein the method attracts animals.

17. The method of claim 14, wherein the method deters animals.

18. The method of claim 14, wherein the method is used in a paintball application.

19. The method of claim 14, wherein the method is used in a BB application.

20. The method of claim 14, wherein the method is used in a marking pellet application.

21. A rupturable capsule comprising:
a capsule containing a composition comprising:
a bioorganic filler; and
silica.

22. The capsule of claim 21, wherein the composition further comprises an oil.

23. The capsule of claim 21, wherein the composition further comprises at least one of a colorant, a fragrance, and a combination thereof.

24. The capsule of claim 21, wherein the composition further comprises water.

25. The capsule of claim 21, wherein the bioorganic filler includes at least one of the following: flours, starches, and a combination thereof.

26. The capsule of claim 22, wherein the oil includes at least one of the following: ethers, esters, paraffins, waxes, fats, and a combination thereof.

27. The capsule of claim 21, wherein the silica includes at least one of the following: silicon dioxide, modified hydrous silicates, hydrated alumina silicates, fumed silica, colloidal silica, organic derivates of hydrous magnesium aluminum silicates, precipitated silica, and a combination thereof.

28. The capsule of claim 21, wherein the silica includes precipitated silica.

29. A method of imparting at least one of a color, a fragrance, and a combination thereof to an object comprising:
rupturing a rupturable capsule on the object, the capsule comprising:
a bioorganic filler;
silica;
an oil;
and at least one of a color, a fragrance, and a combination thereof.

30. The method of claim 29, wherein the method decorates clothing.

31. The method of claim 29, wherein the method attracts animals.

32. The method of claim 29, wherein the method deters animals.

33. The method of claim 29, wherein the method is used in a paintball application.

34. The method of claim 29, wherein the method is used in a BB application.

35. The method of claim 29, wherein the method is used in a marking pellet application.
